(19) United States
(12) Patent Application Publication
(10) Pub. No.: US 2022/0088596 A1
(43) Pub. Date: Mar. 24, 2022

(54) MEMBRANE-COUPLED CONTINUOUS SENSING SYSTEMS

(71) Applicant: University Of Cincinnati, Cincinnati, OH (US)

(72) Inventor: Jason Charles Heikenfeld, Cincinnati, OH (US)

(21) Appl. No.: 17/421,614

(22) PCT Filed: May 3, 2019
(86) PCT No.: PCT/US2019/030574

§ 371 Date: Jul. 8, 2021

Related U.S. Application Data
(60) Provisional application No. 62/791,209, filed on Jan. 11, 2019.

(51) Int. Cl. B01L 3/00; G01N 21/64; G01N 33/53; G01N 33/558

(52) U.S. Cl. CPC B01L 3/502715; B01L 3/5023; G01N 21/6428; G01N 2021/6421; G01N 33/558; B01L 2300/0636; B01L 2300/126; G01N 33/5302

(57) ABSTRACT

A device (100) for sensing a first analyte in a biofluid is provided. The device includes a microfluidic component (130) configured to transport a biofluid sample. The device further includes a sensing solution (142) containing one or more probes, wherein the one or more probes are configured to interact with a first analyte in the biofluid. The device further includes a sensor membrane (172) separating the sensing solution (142) from the microfluidic component (130), the sensor membrane (172) configured to allow transport of the first analyte from the microfluidic component to the sensing solution and prevent transport of the one or more probes out of the sensing solution. The device further includes a sensor configured to sense a reaction between the one or more probes and the first analyte in the sensing solution (142).

### BACKGROUND OF THE INVENTION

Biosensing technologies have enormous potential for applications ranging from athletics, to neonatology, to pharmacological monitoring, to personal digital health, to name a few applications. However, one repeated challenge with biosensing systems is development of sensing modalities that work at challenging concentrations of the analytes measured in a biofluid. This challenge is unfortunate and may be unexpected at first glance because benchtop assay technologies already exist for most analytes of interest. However, those benchtop assays are not easily integrated into biosensor device formats, which typically require miniaturization and simplicity not found with conventional benchtop assays.

A second challenge is that most assays are developed for very specific fluid conditions (e.g., pH, salinity, etc.). In biosensor devices, the fluid conditions can vary significantly as determined by biology and other factors. If these issues can be resolved, a greater array of conventional assay technology can be used in biosensor devices.

### SUMMARY OF THE INVENTION

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

Embodiments of the disclosed invention provide membrane-coupled continuous biofluid sensing systems capable of allowing a greater array of conventional assays to be used in continuous biosensing devices. Even new assays may be used in embodiments described herein, benefiting from the inventive aspects disclosed herein.

### BRIEF DESCRIPTION OF THE DRAWINGS

The objects and advantages of the disclosed invention will be further appreciated in light of the following detailed descriptions and drawings in which:

FIG. 1 is a cross-sectional view of a continuous biosensing device according to an embodiment of the disclosed invention with membrane coupling to at least one sensing material or sensing subsystem.

FIG. 2 is a cross-sectional view of a continuous biosensing device according to another embodiment of the disclosed invention with membrane coupling to at least one sensing material or sensing subsystem.

FIG. 3 is a cross-sectional view of a continuous biosensing device according to another embodiment of the disclosed invention with membrane coupling to at least one sensing material or sensing subsystem.

### DEFINITIONS

As used herein, “biofluid” means a fluid source of analytes. For example, sweat is a biofluid source of analytes that is from eccrine or apocrine glands. For another example, a biofluid could be a solution that bathes and surrounds tissue cells such as interstitial fluid. Embodiments of the disclosed invention may focus on interstitial fluid found in the skin extracted through microneedles and, particularly, interstitial fluid found in the dermis. Biofluid could also include blood, saliva, tears, or other possible biofluids.

As used herein, “continuous monitoring” means the capability of a device to provide at least one measurement of a biofluid, determined by a continuous or multiple collection and sensing of that measurement or to provide a plurality of measurements of that biofluid over time. For example, a sensor could repeatedly sense analytes coming from a continuous stream of biofluid with analytes (e.g., multiple measurements). For example, a sensor could sense analytes coming from a biofluid stream that flows for a long enough duration or enough times of repeated flow such that the sensor is able to reach its proper signal (e.g., single measurement achieved by continuously collecting analyte from a continuous flow of biofluid with the analyte).

As used herein, “measured” can imply an exact or precise quantitative measurement and can include broader meanings such as, for example, measuring a relative amount of change of something. Measured can also imply a binary measurement, such as ‘yes’ or ‘no’ type measurements.

As used herein, “advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

As used herein, “convection” is the concerted, collective movement of groups or aggregates of molecules within fluids and rheids, either through advection or through diffusion or a combination of both.

As used herein, “reversible sensor” means the sensor is able to measure both increasing and decreasing concentrations without any additional change in stimulus or environment for the sensor other than the change in the analyte concentration.

### DETAILED DESCRIPTION OF THE INVENTION

The embodiments described herein will be primarily, but not entirely, limited to wearable sweat sensing devices, and methods or sub-methods using wearable sweat sensing devices. The disclosed embodiments may be practiced using any type of wearable sweat sensing device that measures sweat, sweat generation rate, sweat chronological assurance, sweat solutes, solutes that transfer into sweat from skin, a property of or things on the surface of skin, or properties or things beneath the skin. A sweat sensing device as discussed herein can take on many forms, including patches, bands, straps, portions of clothing or equipment, or any suitable mechanism that reliably brings sweat stimulating, sweat collecting, and/or sweat sensing technology into intimate proximity with sweat as it is generated.

Certain embodiments of the disclosed invention show sensors as simple individual elements. It is understood that many sensors may utilize two or more electrodes, reference electrodes, or additional supporting technology or features that are not captured in the description herein for the sake of simplicity. Sensors are preferably electrical in nature, but may also include optical, chemical, mechanical, or other biosensing mechanisms. Sensors can be in duplicate, triplicate, or more, to provide improved data and readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components are not explicitly shown in the diagrams or described in the embodiments of the disclosed invention. Additionally, descriptions of elements in the alternative may be considered as distinct alternative embodiments that are exclusive of one another. Further, the specific embodiments have distinct combinations of elements, but these elements may be incorporated across embodiments shown. Likewise, the advantages disclosed for an embodiment may apply equally to other embodiments.

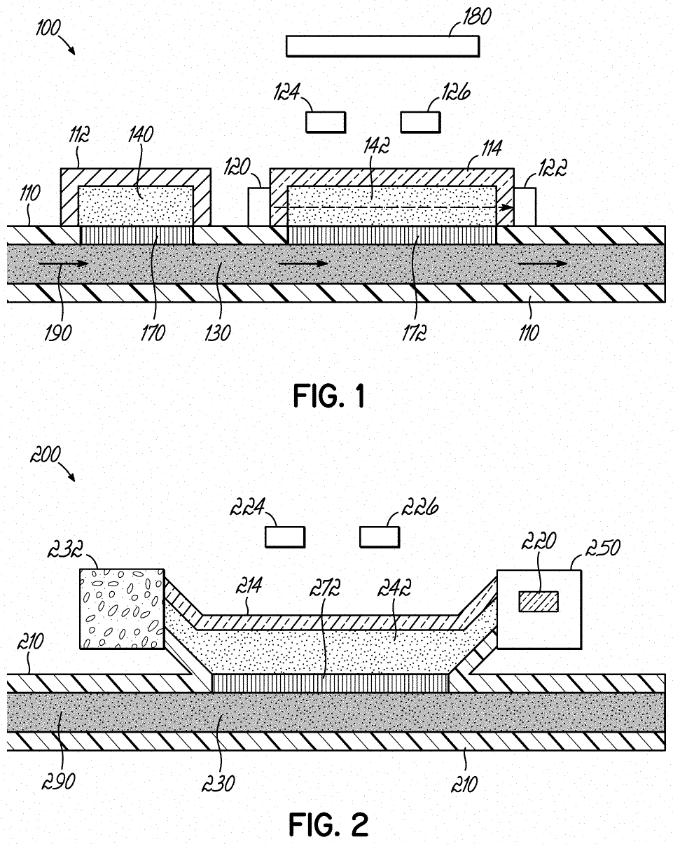

With reference to FIG. 1, which depicts a representative biosensing system comprising a device 100 to which the present disclosure applies. The device 100 is capable of detecting an analyte of interest included in a sample. The device 100 includes a fluid-impermeable substrate 110 adjacent to a microfluidic component 130. The device 100 may be placed near or directly on a wearer's skin. Alternatively, the device 100 may be worn as a mouthpiece within a wearer's mouth.

The microfluidic component 130 is fluidically connected to the wearer's skin or mouth. During operation of the device 100, biofluid from the wearer is transported to the microfluidic component 130. The microfluidic component 130 is capable of advectively transporting a sample stream of biofluid in a direction indicated by arrow 190 from the wearer. The substrate 110 may be made of, for example, a polymer, such as polyethylene terephthalate (PET). The microfluidic component 130 may be, for example, a wicking material, such as paper, a microfluidic channel that is empty before fluid is driven into the channel by wicking force or with pressure driven flow (e.g., syringe pump), or another suitable microfluidic component. A polymer or metal housing 112 together with a buffer membrane 170, surrounds a buffer 140 which could include a buffering solute, such as citrate.

The buffer membrane 170 separates the buffer 140 from the microfluidic component 130. The buffer membrane 170 is coupled to or embedded within the substrate 110, and the buffer membrane 170 is in fluidic communication with the microfluidic component. The buffer membrane 170 may be made of a dialysis membrane or other material that allows transport of solutes in a predetermined molecular weight range. The solutes which may be allowed to transport across the buffer membrane 170 may be solutes that affect pH and salinity of the biofluid present in the microfluidic component 130. As the biofluid sample travels in the direction of the arrow 190 in the microfluidic component 130 adjacent to the buffer membrane 170, the sample is buffered via diffusion of solutes from the buffer 140 through the buffer membrane 170 (described further below). In an embodiment, the buffer 140 could have a volume that is at least ten times greater than the volume of a portion of the microfluidic component 130 in fluid communication with the buffer, for example the portion of the microfluidic component 130 bordering the buffer membrane 170, or at least ten times greater than the volume of the biofluid sample flowing through the device 100. The buffer 140 may contain a first solute composition configured to affect a property, such as pH or salinity, of the biofluid flowing through the microfluidic component. The relatively large volume of buffer 140 compared to the volume of the portion of the microfluidic component 130 is advantageous at least because the relatively large volume extends the useful lifetime of the buffer 140. The biofluid sample stream indicated by arrows 190 may have a second solute composition, different from the solute composition of the buffer 140. The biofluid sample stream is buffered by the first solute composition by diffusion of solutes through the buffer membrane 170 such that the biofluid has a predetermined value for pH or salinity.

Still referring to FIG. 1, downstream from the buffer membrane 170 is a sensor membrane 172. The sensor membrane 172 is coupled to the substrate 110 and is in fluidic communication with the microfluidic component 130. The sensor membrane 172 separates the biofluid sample from a sensing solution or sensing material 142. The sensor membrane 172 may also be made of a dialysis membrane, osmosis membrane, nanofiltration membrane, or other material that allows transport of the target analyte to pass through the sensor membrane 172 from the biofluid sample to the sensing solution 142. The sensing solution 142 contains at least one probe (e.g., an aptamer (not shown)) specific to at least one analyte in the biofluid stream. Because the probes may work best in an optimal pH and salinity range, the sample may be buffered by the buffer 140 prior to reaching the sensor membrane 172. The buffering of the sample stream is optional and may not be needed for all types of probes. The sensing solution 142 may be, for example, a hydrogel and is enclosed by a housing 114. The housing 114 may be transparent (e.g., glass or acrylic) to allow for optical sensing mechanisms as described below.

In some examples, the device is stored in a dry state with probes initially in a dry state. Once biofluid is first introduced into the device 100, water, for example, may pass through sensor membrane 172 and form an aqueous solution with the probes to form the sensing solution 142.

In some examples, the sensor membrane 172 may have a molecular weight cutoff of at least one of less than 100,000 Da, less than 30,000 Da, less than 10,000 Da, less than 3000 Da, less than 1000 Da, or less than 300 Da. A molecular weight cutoff means as used by standard in the industry of membranes, and is a cutoff above which analytes or solutes are unable to significantly traverse the membrane (i.e., analytes or solutes above the molecular weight cutoff are significantly blocked by the membrane).

With further reference to FIG. 1, the device 100 includes components configured to sense interactions occurring in the sensing solution 142 between the probes and the analyte(s) of interest. In an embodiment where the probes are functionalized to allow fluorescent reporting, a fluorescent emission may be detected using a light emitting diode (LED) excitation source 124 and a fluorescence detector 126. In an embodiment, the LED excitation source 124 may emit light that contacts the probes. As a result of the contact of the light emitted from the LED excitation source 124, the probes transmit an amount of light having a fluorescence. The magnitude of the fluorescence is measured by the fluorescence detector 126. The level of fluorescence measured by the fluorescence detector 126 correlates to an analyte concentration in the sensing solution 142.

The device 100 may be configured to allow multiplex detection of two or more analytes. For example, sensing solution 142 could also contain two or more probes, each with distinct fluorescent properties. The probes may be calibrated to produce fluorescence in response to two different analytes, and the fluorescence detector 126 could be a spectrometer with the ability to determine the fluorescent intensities and/or colors transmitted by each probe. Similar to the process described in the previous paragraph, the LED excitation source 124 may emit light that contacts the probes, resulting in the probes producing a fluorescence based on the analyte concentration in the sensing solution 142. Although an ultraviolet, violet, blue, or white light-emitting diode 124 and visible spectrum spectrometer 126 can differentiate multiple wavelengths, in some cases, multiple source 124 and detection 126 systems may be needed, one for each wavelength, for example by using narrow spectrum lasers 124 and photodiode detectors with narrow band-pass optical filters.

Additionally, or alternatively, an LED source 120 and a detector system 122 allow for colorimetric detection. The LED source 120 may be coupled to a side or surface of the transparent housing 114. Aptamer sensing approaches, such as those relying on gold-nanoparticles, are generally less reversible (i.e., suitable for continuous sensing) than fluorescent-based aptamer sensing approaches. The LED source 120 directs light through the transparent housing 114 and through the sensing solution 142. The detector system 122 may be coupled to a side of the transparent housing opposite the LED source. The detector system 122 detects the light after it passes through the opposite side of the transparent housing 114 and can analyze the absorption of light as a signal output. In another embodiment, an external sensor 180, such as a smart phone or other external sensor, may be used to sense the analyte(s) of interest. A smart phone with a light, camera, and software could take photos of the sensing solution 142 though the housing 114 and use the color or fluorescence of the sensing solution 142 to extract measurement data from the device 100.

An example application of the device 100 is to continuously sense cortisol in a biofluid sample. The buffer membrane 170 could be a dialysis membrane with a molecular weight cutoff of between 100 Da and 200 Da, inclusively, which will allow buffering of salinity and pH. For example, the sample stream may enter the device 100 at a pH of 5 and a salinity of 100 mM and then be buffered to a pH of 7 and a salinity of 10 mM, which is within the range of optimal operating conditions for the aptamers in the sensing solution 142. In an example, an analyte of interest in the sample stream is cortisol (about 400 Da), which is too large to be buffered (i.e., cannot diffuse through buffer membrane 170), for example, when the membrane has a rating of less than 100 Da. The buffered sample stream is then transported further through the microfluidic component 130 where it encounters the sensor membrane 172. The sensor membrane 172 could be a dialysis membrane with a molecular weight cutoff of 5000 Da. The sensor membrane 172 allows analytes, including the cortisol, to be transported into the sensing solution 142. The probes in sensing solution 142 could be, for example, aptamers specific to cortisol with a molecular weight of greater than 15,000 Da. The probes are larger than the molecular weight cutoff for the sensor membrane 172 such that they are not able to substantially diffuse through the sensor membrane 172. The aptamers could be functionalized with known reporting techniques, such as a fluorescent tag and a quencher. As cortisol binds with the aptamers, they undergo a shape conformation change that quenches their green fluorescent emission, as detectable by the combination of a blue LED excitation source 124 and a green fluorescence detector 126. Because aptamers are reversible, as cortisol concentration decreases in the sample stream, the fluorescent signal would increase. As a result, a continuous measurement of cortisol concentration in the sample stream is achieved. As described above, the device 100 may be configured to allow multiplex detection of two or more analytes. For detection of cortisol and vasopressin (1,000 Da), the sensing solution 142 could also contain a red fluorescent probe for vasopressin, and the detector 126 could be a miniature spectrometer that is able to discriminate the intensity of both red and green fluorescent intensities. An example can be taught as follows. A biofluid sample starts flowing through the microfluidic component 130. The biofluid sample has a vasopressin concentration of X, and the sensing solution 142 has a vasopressin concentration of 0. Vasopressin passes through the sensor membrane 172 over time until the concentration in the solution 142 has a concentration of X. If the concentration of vasopressin in the flowing biofluid sample changes to X+1, more vasopressin will enter the solution 142, lowering the fluorescent signal. If the concentration of vasopressin in the flowing biofluid sample changes to X−1, vasopressin will leave the solution 142 until the concentration is X−1, increasing the fluorescent signal. Depending on the volumes of fluids, sizes of analytes (diffusion coefficients), concentration gradients for the analytes (diffusion velocity), membrane flow/diffusion resistance, and other factors, the response time for the device 100 to a change in concentration of analyte could range from seconds to hours, or even longer, with a preferable response time being on the order of minutes.

With further reference to FIG. 1, the probes could also be multiple fluorescent sandwich-style antibody probes with size greater than 100,000 Da, and therefore the sensor membrane 172 could be made non-porous to solutes greater than 100,000 Da, yet porous to larger analytes such as many cytokine proteins that have molecule weights less than 100,000 Da.

With further reference to FIG. 1, in an embodiment, the device 100 is configured to be insensitive to flow rate of the incoming sample stream by at least less than 20% change of the measured analyte signal for change in flow rate of 2X or more. For example, if the initial flow rate begins as X, the initial measured concentration is Y, and the flow rate later changes to 2X; then the measured concentration stays within 0.8-1.2Y. To achieve this, there must be adequate time for analytes to diffuse through the sensor membrane 172 to reach the sensing solution 142 a concentration close to equilibrium with the concentration in the biofluid sample stream (arrow 190). For example, for a sample stream flow rate of 500 nL/min, a microfluidic component 130 could be 60 μm thick, 2 mm wide, and 5 cm long and be adjacent to sensor membrane 172, and the thickness of the sensing solution 142 in housing 114 could also be 60 μm thick.

With reference to FIG. 2, in an embodiment where like numerals in FIG. 2 correspond with like numerals in FIG. 1, a device 200 is shown that senses an analyte using probes that are not reversible in their detection mechanisms (e.g., antibodies). The device 200 includes a solution 242 that flows through a channel between material 214, substrate 210, and membrane 272. The solution 242 is supplied by a reservoir 232 to the channel and is then collected by an analyzer 250. The analyzer 250 contains at least one sensor 220. As a result, non-reversible probes in solution can be continually advectively introduced from the reservoir 232 and be detected by the sensor(s) 220 in the analyzer 250. The non-reversible probes could also be detected using other techniques, such as having their fluorescence measured by elements 224, 226 through an optically transparent material 214. Reservoir 232 could be a pump that provides solution 242, such as a syringe pump. Additionally, the reservoir 232 could release the fluid upon application of pressure (e.g., a blister pack), and the fluid may be transported to analyzer 250 by a wicking force (e.g., analyzer 250 could contain a wicking hydrogel). A particular advantage for use of antibody-based detection methods is that antibody-based detection methods generally include lower limits of detection compared to aptamers.

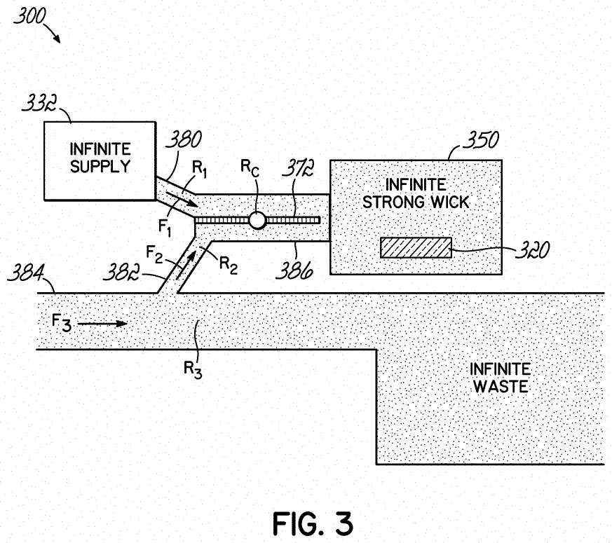

With reference to FIG. 3, in an embodiment where like numerals in FIG. 3 correspond with like numerals in FIGS. 1 and 2, a device 300 is shown that senses an analyte using probes that are not reversible in their detection mechanisms (e.g., antibodies). The device 300 includes a solution reservoir 332 that supplies a solution that flows through a solution channel 380 having a radius R₁, and solution channel 380 has a flow rate F₁. A sample of the fluid of interest enters a sample channel 382, which has a radius R₂, from a passage 384, which has a radius R₃. The flow rate of the fluid in the passage 384 F₃ and R₃ are significantly higher than the flow rate of the sample entering the sample channel 382 F₂ and R₂, respectively. Flow rates F₁ and F₂ could be similar or different, depending on the kinetics of the assay strategy used. For example, if a competitive binding assay is utilized, flow rates could be adjusted such that the device would operate with a wide dynamic sensing range at the concentration of the analyte in the sample channel 382. For example, if the analyte had very strong binding kinetics to one or more probes or antibodies in the solution channel 380, then flow rate Fi could be faster (e.g., even 10 or more times faster) than flow rate for the sample channel 382 F₂. Along a portion of each, the solution channel 380 and the sample channel 382 are adjacent one another in a sensing channel 386, with a membrane 372 with a fluid or diffusion resistance R_{c}. In the sensing channel 386, the solution channel 380 and the sample channel 382 are separated by a membrane 372. The sensing channel 386 is fluidically coupled to an analyzer 350 configured to provide a strong wicking force, which analyzer includes at least one sensor 320. As a result, non-reversible probes in solution can be continually advectively introduced from the reservoir 332 and be detected by the sensor(s) 320 in the analyzer 350. The non-reversible probes could also be detected using other techniques, such as those described above. The sample channel 382 is sized to create a flow rate slow enough that no significant water flux is needed. Minimal water flux may occur due to salt equilibrating through the membrane 372 (i.e., due to osmosis).

With further reference to FIGS. 2 and 3, in an aspect of the disclosed invention, surface bound probe chemistries may be used (e.g., an antibody or aptamer based probe will bond with the substrate and become immobilized). Such bound probe chemistries or assays can be regenerated, e.g., by changing pH to cause the probes to release the analyte. However, in some cases, the probes themselves will need to be released from a surface (e.g., material 214). Consider an embodiment where material 214 is transparent and is coated with a transparent indium-tin-oxide electrode. Voltage could be applied to such an electrode to create electrolysis, change pH, and/or etch the indium-tin-oxide such that the probes are released from the surface. As a result, an electrochemical based process is used to regenerate the electrode after use.

In an alternate embodiment, probes such as fluorescent aptamers could be immobilized in a hydrogel (linked to the hydrogel structure itself) possibly eliminating the need for membrane coupling. For example, the quencher or fluorescently tagged end of the aptamer could have a functional group that bonds to agar, gelatin, or other hydrogel structures.

While the miniaturization provided by aspects of the disclosed invention is valuable to applications involving biofluids, embodiments of the present invention may also be useful in applications involving non-biofluids (e.g., river pollution).

While specific embodiments have been described in considerable detail to illustrate the disclosed invention, the description is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope of the general inventive concept.

1. A device comprising:
a microfluidic component configured to transport a biofluid sample;
a sensing solution containing one or more probes, wherein the one or more probes are configured to interact with a first analyte in the biofluid;
a sensor membrane separating the sensing solution from the microfluidic component, the sensor membrane configured to allow transport of the first analyte from the microfluidic component to the sensing solution and prevent transport of the one or more probes out of the sensing solution; and
a sensor configured to sense a reaction between the one or more probes and the first analyte in the sensing solution.

2. The device of claim 1, further comprising:
a buffer including one or more buffering solutes; and
a buffer membrane separating the buffer from the microfluidic component, the buffer membrane configured to allow transport of the one or more buffering solutes into the microfluidic component and to prevent the first analyte from entering the buffer.

3. The device of claim 1, wherein the microfluidic component comprises at least one of a wicking material or a microfluidic channel.

4. The device of claim 3, wherein the microfluidic component comprises the wicking material, and the wicking material comprises paper.

5. The device of claim 2, wherein the buffer has a volume at least ten times greater than a volume of a portion of the microfluidic component that is in fluidic communication with the buffer.

6. The device of claim 2, wherein the buffering component has a volume at least ten times a volume of the biofluid present in the device.

7. The device of claim 1, wherein the sensor membrane borders the microfluidic component.

8. The device of claim 1, wherein the sensing solution comprises a hydrogel.

9. The device of claim 1 further comprising:
an excitation source positioned to direct light to the one or more probes; and
a fluorescence detector, positioned to measure a magnitude of light transmitted from the one or more probes, wherein the magnitude of light measured by the fluorescence detector correlates to an analyte concentration in the sensing solution.

10. The device of claim 9, wherein the one or more probes include a first probe calibrated to produce, from the light emitted by the excitation source, a first fluorescence corresponding to a first analyte, and a second probe calibrated to produce, from the light emitted by the excitation source, a second fluorescence corresponding to a second analyte, the first analyte being different from the second analyte.

11. The device of claim 10 wherein the fluorescence detector comprises a spectrometer configured to differentiate the first fluorescence from the second fluorescence.

12. The device of claim 1 further comprising:
a light source coupled to a first side surface of a transparent housing, the transparent housing enclosing the sensing solution; and
a detector system coupled to a second side surface of the transparent housing, wherein the second side surface is located opposite the transparent housing from the first side surface,
wherein the light source is configured to transmit light through the transparent housing, and through the sensing solution, and to be detected by the detector system.

13. The device of claim 1, wherein the sensor membrane is nonporous to solutes having a molecular weight greater than 100,000 Daltons.

14. The device of claim 1, wherein the sensor membrane has a molecular weight cutoff of less than 100,000 Da, less than 30,000 Da, less than 10,000 Da, less than 3000 Da, less than 1000 Da, or less than 300 Da.
